(19) United States
(12) Patent Application Publication
(10) Pub. No.: US 2006/0032506 A1
(43) Pub. Date: Feb. 16, 2006

(54) Apparatus and method for adapting word processing software and adjusting printers to deliver via internet prophylactic sizing kit to remote site, to correlate measurements with prophylactic sizes to make order, to remit correlated information to point of sale

(76) Inventor: Frank C. Sadlo, Las Vegas, NV (US)

Correspondence Address: TOD R. NISSLE, P.C., P.O. Box 55630, Phoenix, AZ, 85078, US

(21) Appl. No.: 10/913,761

(22) Filed: Aug. 6, 2004

(51) Int. Cl. A61F 6/04

(52) U.S. Cl. 128/844

(57) ABSTRACT

An apparatus and method are provided to adapt word processing software and printers to enable a prophylactic sizing kit to be delivered via the Internet to a remote site, to correlate penile measurements with prophylactic size to make or order, and to remit correlated order information to a point of sale.

This invention pertains to an apparatus and method for obtaining a prophylactic.

More particularly, the invention pertains to an apparatus and method for providing a prophylactic sizing kit, for delivering the prophylactic sizing kit to a location remote from a point of sale of prophylactics, and for utilizing the prophylactic sizing kit to select a prophylactic to obtain from the point of sale.

In my U.S. Pat. No. 5,857,466, I describe apparatus for customizing a prophylactic. The apparatus provides one or more scales to measure the width and length of an erect penis to obtain a prophylactic that closely fits the erect penis. The apparatus has important advantages in that it is believed to significantly reduce the risk that the prophylactic will not fit, that the prophylactic will come off during intercourse, that the prophylactic could injure the user, and that the user will contract a sexually transmitted disease.

The idea of utilizing a scale or scales to measure an erect penis seemed appropriate at the time the patent application that matured into U.S. Pat. No. 5,857,466 was filed. However, putting such into common practice was a problem for several reasons. First, most prophylactic users are accustomed to making a decision on which condom to purchase at the point of sale, i.e., in the store, are often interested in using the condom within a relatively short period of time and are not interest in taking penile measurements, and can not, practically speaking, accomplish the measurement of an erect penis at the point of sale. Second, some prophylactic users can not read or are not readily able to follow printed instructions. Third, an effective means of delivering the scales to measure an erect penis did not exist. Fourth, the measuring scales have to maintain, when processed by a printer or computer program or web site program, dimensional integrity, otherwise the prophylactic obtained by a user may not be the suggested size of prophylactic intended by the measurement system of the invention and the prophylactic may not fit properly. Fifth, the measurement scales have to be provided in a manner that facilitates their use. Placing the scale at a point of sale is believed sometimes to have limited value because users often are embarrassed and are not interested in acquiring such scales while in public. Sixth, there was no existing system to facilitate the correlation of the actual measurements of an erect penis with the measurements of an appropriate condom.

Accordingly, it would be highly desirable to provide an improved method and apparatus to enable a user to measure an erect penis and to obtain a customized prophylactic that better fits and conforms to at least a portion of the erect penis.

Therefore, it is a principal object of the invention to provide an improved method and apparatus for obtaining a better fitted condom.

Another object of the invention is to provide an improved method and apparatus for measuring an erect penis to obtain a better fitted prophylactic.

A further object of the invention is to provide an improved method and apparatus for measuring an erect penis and correlating measurement data with available prophylactic sizes to order an appropriately sized prophylactic from a site remote from the point of sale of the prophylactic.

Still another object of the invention is to provide an improved penile measurement that compensates for errors in the dimensional integrity of the scale or scale used during such measurement.

Yet another object of the invention is to provide an improved measurement apparatus that can be utilized to determine a specific prophylactic size and that can be readily reproduced to facilitate the transmission and use of the measurement kit.

These and other, further and more specific objects and advantages of the invention will be apparent from the following detailed description of the invention, taken in conjunction with the drawings, in which:

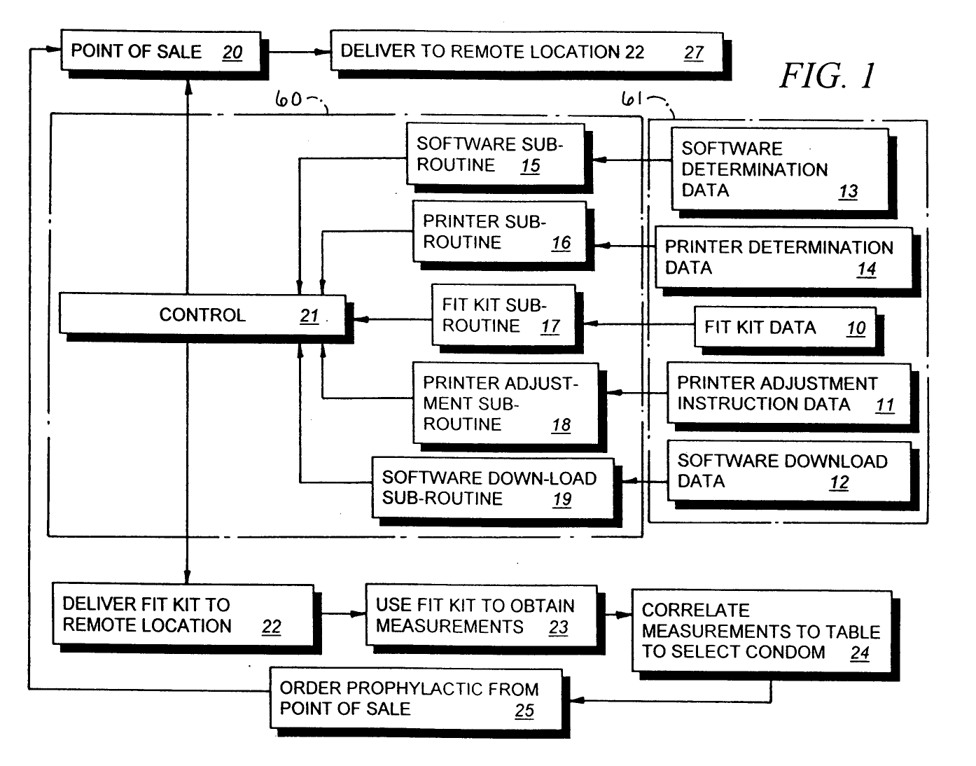

FIG. 1 is a block flow diagram illustrating a system for delivering a measurement kit to a remote site, for using the kit to obtain dimensions of an erect penis, for correlating the dimensions with data to define an appropriately sized prophylactic to order, for ordering the prophylactic from the point of sale, and for delivering the prophylactic from the point of sale to the remote site;

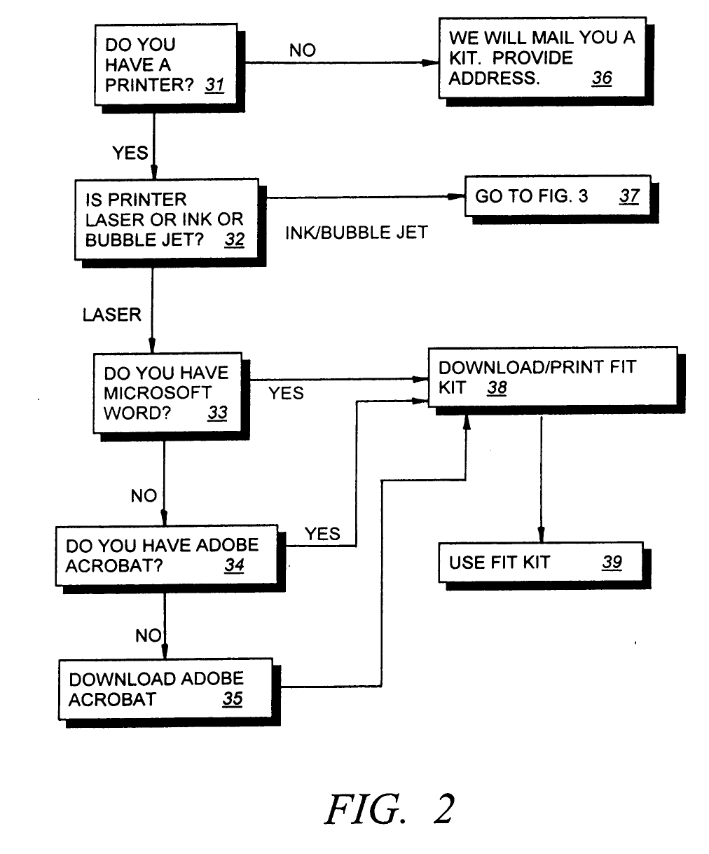

FIG. 2 is a block flow diagram illustrating process steps during use of the system of FIG. 1;

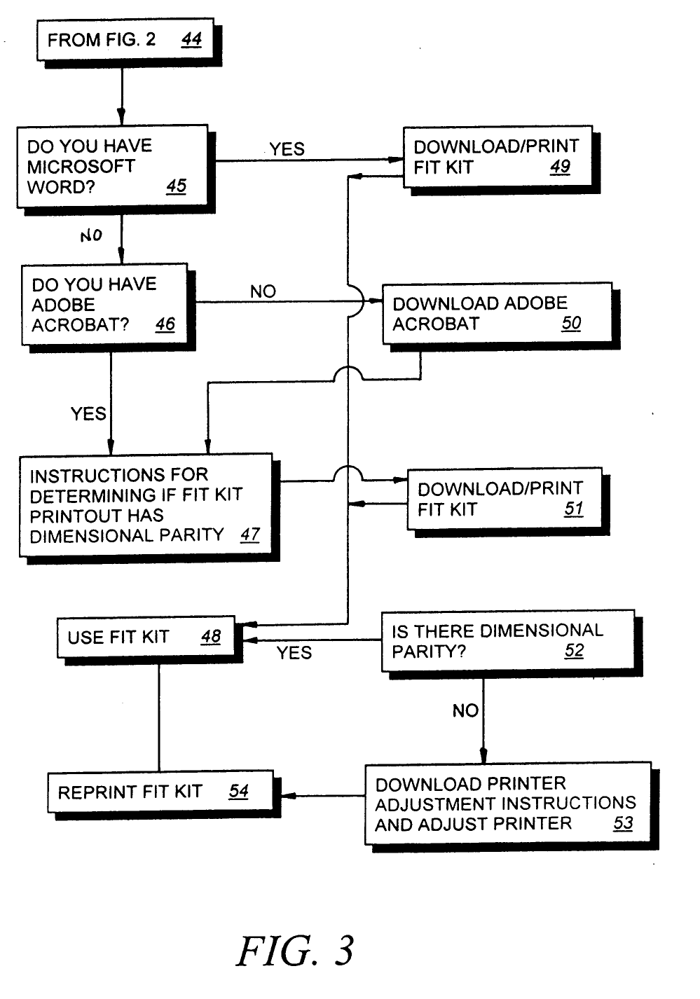

FIG. 3 is a block flow diagram further illustrating process steps during use of the system of FIG. 1;

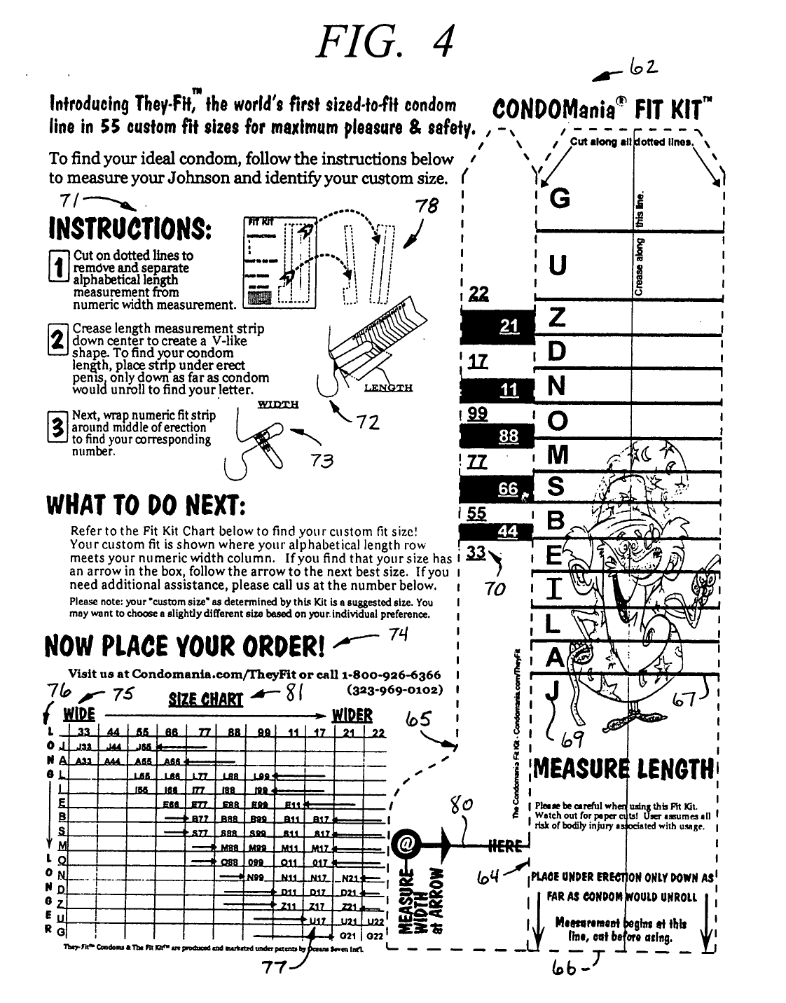

FIG. 4 is an illustration of a measurement fit kit constructed in accordance with the principles of the invention; and,

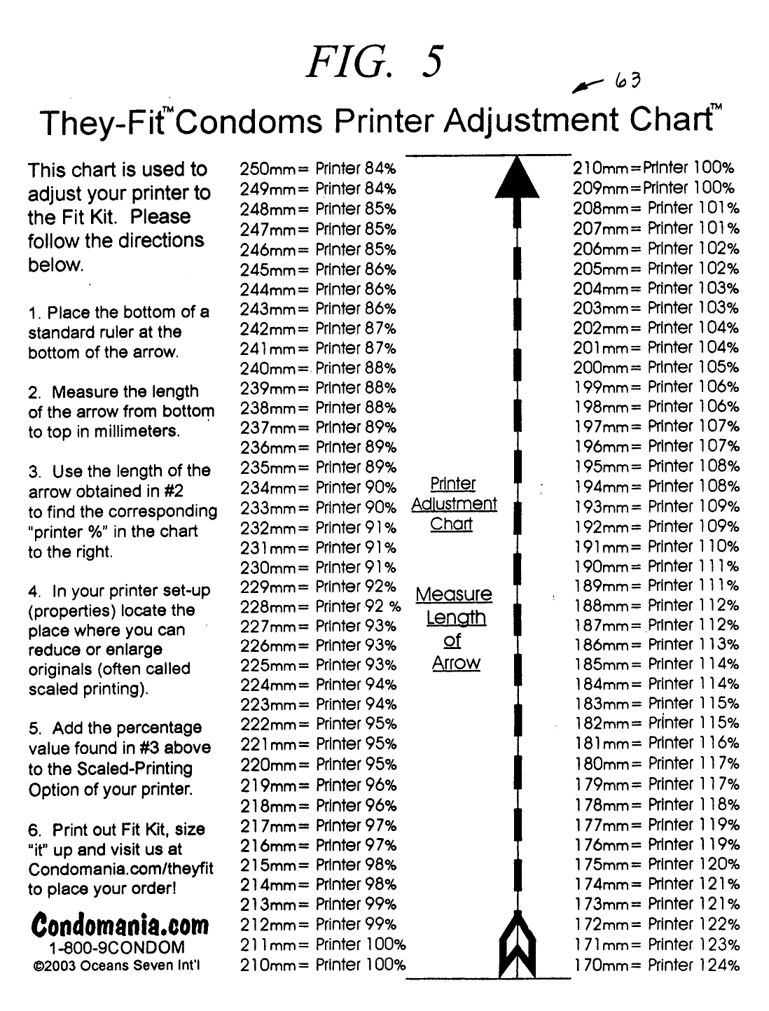

FIG. 5 is an illustration of a printer adjustment chart and instructions constructed in accordance with the principles of the invention.

Briefly, in accordance with my invention, I provide an improved method for delivering by a computer network a prophylactic sizing kit to a site that is remote from a point of sale of prophylactics. The system comprises the step of providing a printable prophylactic sizing kit. The kit includes at least one downloadable scale for measuring at least one of a pair comprising the length and width of an erect penis; instructions for utilizing the scale; and, a downloadable size chart correlating measurements made with the scale with indicia utilized to provide prophylactics of a selected size. The method also includes the step of providing a computer network web site to identify particular word processing software to download the prophylactic sizing kit; to identify a printer that alters the size of the sizing kit when the sizing kit is downloaded from the web site; to provide a downloadable printer adjustment chart when the printer is used to download the sizing kit from the web site; and, to provide downloadable instructions for utilizing the printer adjustment chart. The method also includes the steps of downloading to the remote site from the web site the sizing kit, the printer adjustment chart, the instructions to utilize the scale, and the instructions for utilizing the printer adjustment chart; of utilizing the instructions for utilizing the printer adjustment chart to determine if the scale in the sizing kit is properly sized; and, when the scale is not properly sized, using the printer adjustment chart to adjust the printer to print the sizing kit with a properly sized scale.

In another embodiment of the invention, I provide an improved method for delivering by a computer network a prophylactic sizing kit to a site that is remote from a point of sale of prophylactics and for obtaining a prophylactic from the point of sale. The method comprises the step of providing a printable prophylactic sizing kit including at least one downloadable scale for measuring at least one of a pair comprising the length and width of an erect penis; instructions for utilizing the scale; and, a downloadable size chart correlating measurements made with the scale with indicia utilized to provide prophylactics of a selected size. The method also includes the step of providing a computer network web site to identify particular word processing software to download the prophylactic sizing kit such that said software will maintain the size of the document when the document is downloaded from the web site; and, to identify a printer that will maintain the proper size of said document when the document is downloaded from the web site. The method also comprises the steps of downloading to the remote site from the web site the sizing kit, and the instructions to utilize the scale; utilizing the printer to print the sizing kit; utilizing the instructions to measure at least one of said pair at the remote site; utilizing the size chart to correlate the measurements of at least one of said pair with a prophylactic of a selected size; and, ordering the prophylactic of the selected size from the point of sale.

In a further embodiment of the invention, I provide an improved method for delivering a prophylactic sizing kit to a remote site that is remote from a point of sale of prophylactics and for obtaining a prophylactic from the point of sale. The improved method comprises the step of providing digital data defining a printable prophylactic sizing kit document including at least one scale for measuring at least one of a pair comprising the length and width of an erect penis; instructions for utilizing the scale; and, a size chart correlating measurements made with the scale with indicia utilized to provide prophylactics of a selected size. The method also includes the steps of providing a computer program to download the sizing kit document to a printer such that the software will maintain the proper size of the document when the document is downloaded to the printer; providing a printer that maintains the proper size of the document when the document is printed by the printer; utilizing the computer program to download the sizing kit document to the printer such that the printer prints the prophylactic sizing kit; delivering the printed prophylactic sizing kit to the remote site; utilizing the instructions to measure at least one of a said pair at the remote site; utilizing the size chart to correlate the measurements of at least one of said a prophylactic of a selected size; and, ordering the prophylactic of the selected size from the point of sale.

In another embodiment of the invention, I provide apparatus to deliver a prophylactic sizing kit to a remote site that is remote from a point of sale of prophylactics and for obtaining a prophylactic from the point of sale. The apparatus includes digital data defining a printable prophylactic sizing kit document including at least one scale for measuring at least one of a pair comprising the length and width of an erect penis; instructions for utilizing the scale; a size chart correlating measurements made with the scale with indicia utilized to provide prophylactics of a selected size; a computer program to download the sizing kit document to a printer such that the software will maintain the proper size of the document when the document is downloaded to the printer; a download printer to print said sizing kit document, and maintain the proper size of the document when said document is printed by said printer; a system to deliver the printed prophylactic sizing kit to the remote site; a system to order a prophylactic of a selected size from the point of sale after the sizing kit and the instructions are used to measure at least one of said pair at the remote site, and the size chart is utilized to correlate the measurements of at least one of said pair with a prophylactic of a selected size.

Turning now to the drawings, which depict the presently preferred embodiments of the invention for the purpose of illustrating the practice thereof and not by way of limitation of the scope of the invention, and in which like reference characters refer to corresponding elements throughout the several views, FIG. 1 illustrates a system constructed in accordance with the invention and including a computer with a controller 60 and memory 61. Memory 61 includes software determination data 13, printer determination data 14, fit kit data 10, printer adjustment instruction data 1 1, and software download data 12. Controller 60 includes software sub-routine 15, printer sub-routine 16, fit kit sub-routine 17, printer adjustment sub-routine 18, and software download sub-routine 19. Control 21 communicates as needed with sub-routines 15 to 19 and communicates with point of sale 20 by transmitting orders to point of sale 20, by accepting updates from point of sale 20, etc. Control 21 also communicates 22 over a computer network with a remote location to permit a fit kit, printer adjustment instruction, and other desired data to be delivered to a computer for printing at the remote location. Control 21 also permits communication 22 with a remote location by collecting information from the remote location that permits a penis measurement kit 62 to be mailed or otherwise delivered to the remote location. The remote location typically is a home or business. Once the measurement kit or “fit kit” 62 is delivered 22 to the remote location or to another desired site, the measurement kit 62 is used 23 to obtain measurements of an erect penis. The measurements are correlated 24 to data in a table to select an appropriately sized condom, and the condom is ordered 25 from the point of sale 20. The point of sale 20 receives the order, fills it, and has the order delivered 27 to the remote location or some other site. The point of sale 20 is the location at which the order to ship or deliver the prophylactic is generated. Such an order typically is generated upon receipt of payment for the prophylactic. A retail store is a point of sale because a condom is delivered to a customer upon receipt of payment. The point of sale can be adjacent or can comprise computer 60, 61 or can be remote from computer 60, 61.

The system of FIG. 1 is, by way of example and not limitation, discussed in more detail with reference to FIGS. 2 to 5. In particular, FIGS. 2 to 5 are described in connection with an implementation of the invention using a web site on the Internet or other computer network. When a user accesses the web site and indicates that he wishes to obtain a measurement kit (i.e., a “fit kit”) for measuring an erect penis and ordering a better fitted prophylactic based upon such measurements, the printer sub-routine 16 of controller 60 causes the question 31 “Do you have a printer?” to appear on the web site that is being viewed by the user on the user's computer screen.

If the user answers “NO” (typically by clicking with his mouse on a “no” button that is on the website and is visible on the user's computer or by typing in “no” in a box or field that is on the web site and is visible on the user's computer screen and pressing the “enter” button on his keyboard), then printer sub-routine 16 causes the message “We will mail you a kit. Please provide address.” to appear on the web site (and consequently on the user's computer screen), typically with a grouping of informational boxes that can be completed by the user with his keyboard to provide the user's name and address. Alternatively, the user can be provided with a container that includes a fit kit 62, and if desired, one or more condoms or other articles. The condoms in the container can be the same size or different sizes and the container can be mailed or otherwise delivered or provided to the user. The container can consist of conventional mailing envelopes, boxes, foil packs, or any other means for transmitting something by mail or by any other delivery system.

If the user answers “YES” to question 31, then sub-routine 16 causes the question 32 “Is the printer a laser printer or ink or bubble jet printer?” to appear on the web site.

If the user, by entering appropriate information with his keyboard or by clicking with his mouse on an appropriate answer box or boxes on the web site, answers question 32 by indicating that the printer is an ink printer or bubble jet printer, then, as is indicated by “GO TO FIG. 3” (in FIG. 2) and by “FROM FIG. 2” 44 (in FIG. 3), the controller 60 causes printer sub-routine 16 to “hand off” or transfer control to the software sub-routine 15. Any desired software, hardware, or other technology can, as would be appreciated by those of skill in the art, be used to accomplish the goals and functions of the invention.

Software sub-routine 15 causes the question 45 “Do you have Microsoft Word” to appear on the web site (FIG. 3). If the user answers “YES”, then controller 60 causes sub-routine 15 to “hand off” or transfer control to fit kit sub-routine 17. Sub-routine 17 causes instructions to appear on the web site that enable the user to download the fit kit 62 shown in FIG. 4 so the user can then print 49 the fit kit 62 on his laser printer. After the user prints 49 the fit kit 62 on his printer, he uses 48 the fit kit in the manner later described below. The user, instead of downloading the fit kit 62 to his printer, might elect to download the digital data comprising the fit kit 62 to an electronic or other measuring device that the user can employ to obtain measurements of his erect penis. The measuring device might, by way of example and not limitation, be a hand held device with a laser or other sensing system than can obtain a desired measurement or measurements of an erect penis. Once the measuring device obtains the desired measurement(s), it automatically correlates the measurements with chart data stored in the memory of the device and indicates on a display the particular suggested condom that the user should order. Or, the measuring device provides the user with the measurement data and the user utilizes a chart 81 to determine the suggested condom that should be ordered.

If the user answers question 45 “NO”, sub-routine 15 causes the question 46 “Do you have Adobe Acrobat?” to appear on the web site. If the user answers “NO”, controller 60 causes sub-routine 15 to transfer control to software download sub-routine 18. Sub-routine 18 causes information to appear on the web site that the user utilizes to download Adobe Acrobat software from the web site to the user's computer. After the download of the Adobe Acrobat software is completed, controller 60 causes sub-routine 19 to hand-off control to printer adjustment sub-routine 18. Sub-routine 18 causes instructions 47 to appear on the web site on how to determine if the dimensional parity of the fit kit is maintained when the fit kit is printed by the user's ink or bubble jet printer. The dimensional parity of the fit kit is maintained if the printer (or the user's computer) do not function to enlarge or shrink the fit kit 62. One way to instruct the user to check dimensional parity of the fit kit 62 printed by the user's printer is to measure the distance from the horizontal line between the letters “A” and “J” to the horizontal dashed line 66 near the bottom of the printed page. This distance presently should be 80 mm, plus or minus one mm. If the distance is less than 80 mm or greater than 80 mm by more than one millimeter, then the user's computer and printer did not maintain dimensional parity.

The last portion of the instructions 47 direct the use to download 51 the fit kit by clicking on a link included in instructions 47 and by then following the instructions that appear after the user clicks on the link. After the user downloads 51 the fit kit 62 and prints kit 62 on his printer, the user utilizes the instructions 47 noted above to determine 52 if the downloaded fit kit 62 has dimensional parity. If the downloaded fit kit 62 has dimensional parity, the user utilizes 48 the fit kit. If the downloaded fit kit 62 does not have dimensional parity, the user clicks on a link provided on the web site and then follows the instructions that sub-routine 18 causes to appear on the web site after the user clicks on the link. The instructions enable the user to download 53 from the web site the printer adjustment instructions 63 (FIG. 5). The user utilizes the printer adjustment instructions to adjust his ink or bubble jet print, reprints 54 the fit kit 62 on his ink or bubble jet printer, and the uses 48 the fit kit 62. The printer adjustment instructions are critical in the practice of the invention because the instructions enable the user to compensate for changes in proportion and size produced by the user's printer.

Returning to FIG. 2, if the answer to the question 32 “Is printer laser or ink or bubble jet?” is “Laser”, then the controller 60 causes printer sub-routine 16 to “hand off” or transfer control to the software sub-routine 15. The software sub-routine 15 causes the question 33 “Do you have Microsoft Word?” to appear on the web site. If the user answers question 45 “NO”, sub-routine 15 causes the question 46 “Do you have Adobe Acrobat?” to appear on the web site. If the user answer question 46 “NO”, sub-routine 15 instructs the user to “Download Adobe Acrobat” 35 by clicking on a link listed on the web site. The user clicks on the link to download Adobe Acrobat to the user's computer. The controller 60 then causes sub-routine 15 to transfer control to the fit kit sub-routine 17 and sub-routine 17 permits the user to download 38 the fit kit. The user then prints and uses 39 the fit kit 62.

If the user answers question 34 “YES”, the controller then causes sub-routine 15 to transfer control to the fit kit sub-routine 17 and the sub-routine 17 permits the user to download 38 the fit kit 62. The user then prints and uses 39 the fit kit.

If the user answers question 33 “YES”, the controller then causes sub-routine 15 to transfer control to the fit kit sub-routine 17 and the sub-routine 17 permits the user to download 38 the fit kit. The user then uses 39 the kit.

Use of the fit kit 62 is described with reference to FIG. 4. Advantages of the fit kit of FIG. 4 are that it is printed on a single sheet, it includes each scale 64, 65 that is used to measure an erect penis, it includes written and illustrated instructions for using the scales 64 and 65, it includes a size chart for correlating erect penile measurements with the “flat” dimensions of available condoms to select a prophylactic based on the measurements, and it includes instructions for placing an order.

On printing out the fit kit 62 or otherwise obtaining the fit kit 62, the user cuts out the scales 64 and 65 as indicated in step 1 of the instructions 78. As indicated in step 2 of the instructions 78, the user utilizes scale 64 to determine the length of an erect penis, by placing dashed line 66 (i.e., the bottom of scale 64) at the base of the penis and determining in which rectangular section J, A, L, I, E, B, S, etc. the tip of the penis is located. As indicated in step 3 of the instructions, the user utilizes scale 65 to measure the girth of an erect penis, by wrapping scale 65 around the penis at the midpoint of the length of the penis and by seeing where one of the numbered sections 33, 44, 55, 66, 77, etc. is in registration with the “measure width of arrow” line 80. As would be appreciated by those of skill in art, the indicia or other markings or symbols or coloring or other representations used to designate different locations along a scale 64,65 can be selected and used as desired. An alternate method of measuring the “girth” of an erect penis is, if desired, to simply measure the actual width (instead of the circumference) of the penis. The actual width is the distance from one side of the penis to the other side of the penis. If, for example, an erect penis is assumed to have an middle portion that is cylindrical in shape, the actual width of the middle portion penis would equal the diameter of the cylindrical middle portion. If a erect penis his a somewhat oval shape or irregular shape, the actual width is the greatest side-to-side distance that can be measured.

The girth of an erect penis can, if desired, be measured at a location other than the midpoint of the penis, and more than one measurement of the girth can be taken. For example, in order to select a better fitting condom, it might be desirable to measure the girth of an erect penis at the midpoint of the length of the penis and at or near the tip of the penis or at the base of the penis or at any other points along the length of the penis. Similarly, the length of an erect penis does not have to be measured from the base of the penis. For example, a user might elect to measure the length of an erect penis from a location that is located on the penis ten millimeters from the base of the penis toward the tip of the penis, or from a point located on the underside of the penis or from a point located on a side of the penis. As used herein, the width of a penis is obtained either by measuring the girth or actual width of the penis.

Once the user has obtained measurements of the length and girth of an erect penis, he refers to the size chart 81 to determine the prophylactic to order. For example, if the length of the erect penis is indicated by section U of scale 64 (since the tip of the erect penis was located in section U when scale 63 was positioned adjacent the penis in the manner indicated in section 2 of the instructions 71) and if the girth of the erect penis is indicated by section 17 of scale 65 (because section 17 was in registration with line 80 when scale 65 was wrapped around the middle of the penis in the manner indicated in section 3 of the instructions 71), then the proper prophylactic to order is indicated in the size chart 81 by the indicia U17. If the length of the erect penis is indicated by section L of scale 64 and the girth by section 66 of scale 65, then according to the size chart 81 the proper prophylactic to order is indicated by the indicia L66. The fit kit 62 also provides a system for the user to order a prophylactic. One ordering system comprises visiting a web site: condomania.com. Another ordering system is to call a telephone number and order: 1-800-926-6366. Any other desired ordering system (by mail, by fax, etc.) can be specified in the fit kit 62 or in other materials accompanying kit 62.

The condom in chart 81 that corresponds to the measurements of penile girth and length made by a user is the suggested size condom. This suggested size condom likely will have a better fit and provide more comfort and pleasure than generic condoms. After, however, the user utilizes the suggested size condom, he may elect to utilize a larger or smaller condom depending on his physiology or other factors.

A critical component of the measurement kit 62 of the invention is the utilization of correlation coefficients or values in the preparation of the size chart and the selection of the appropriate condom for a user. A user may have an erect penis with a length that is equal to the distance from dashed line 66 to line 67, which distance equals 80 mm. The girth of the erect penis may be indicated by the distance from line 80 to the bottom of section 44 because that is where section 44 overlaps line 80 when scale 65 is wrapped around the center of an erect penis in the manner illustrated in section 3 of the instructions. The distance from line 80 to the bottom of section 44 equals 85 mm. Consequently, the length of the user's erect penis is 80 mm and the girth is 85 mm. Importantly, this does not mean that the proper condom for the user is 80 mm long and has a girth of 85 mm. It is currently preferred that the condom be one or two mm longer than necessary to insure that the condom is long enough. This means that one correlation coefficient that is used to arrive at the appropriate condom size is multiplied times the length of an erect penis to obtain the desired length of a properly fitted condom. If the correlation coefficient is 1.02 and the length of the erect penis is 80 mm, then the desired length of the condom is 81.6 mm, or 82 mm. Any desired criteria can be utilized to determine a desired correlation coefficient, including, for example, the simple criteria that the condom be two mm longer than the actual measured length of an erect penis, in particular because the bead at the open end of the condom can catch on an anatomical area where the erect penis is being inserted. A condom that is too short increases the likelihood that the condom will come off due to the bead being more susceptible to being pulled along the erect penis toward the end of the penis. Another criteria can be related to the “hardness” or elasticity of the latex of other material used to make the condom. If the condom is made from latex that is relatively soft and elastic, it may be desirable for the width of the condom to be reduced further to compensate for the increased elasticity. Or, if the condom is made from a latex that is relatively hard and not that elastic, it may be desirable for the width of the condom to not be reduced as much compared to the softer latex condom. It is preferred that the girth of the condom be less than the girth of the erect penis such that the condom will expand elastically and tend to frictionally better adhere to the erect penis. The width and length of a condom are normally measured when the condom is laid on a flat surface; hence the width of a condom is called the “flat width” and the length of the condom is called the “flat length”. As a result, the correlation coefficient that is used in connection with determining the width of a properly fitted condom must take into account that the width of the condom must be less than the actual width of the erect penis and that the circumferential girth of the erect penis is a different measurement than the width of a condom. In cases where the girth, and not the width, of an erect penis is measured, the girth measurement must be somehow reconciled and correlated with the “flat width” measurement of a condom when selecting an appropriately sized prophylactic. One simple way of reconciling or correlating the two measurements in accordance with the invention is to assume that the middle portion of an erect penis is cylindrically shaped and to use the mathematical formula that permits the diameter of a cylinder to be determined when the circumference of the cylinder is known. If the circumference of a cylinder is 85 mm, then the diameter is about equal to 85/3.14 or about 27 mm. Once the width (diameter) of the cylinder is known, then an additional second correlation coefficient is used to determine the desired width of the condom. If this second correlation coefficient equals 0.8, then the desired width of the appropriate condom equal (27 mm×0.8)=22 mm. Any desired criteria can be utilized to determine the value of the second correlation coefficient, including but not limited to the elasticity of the latex or other material used to make the condom, the thickness of the condom wall, etc. The correlation coefficients discussed above are incorporated in the size chart 81 so that a condom selected using chart 81 normally has a width that is less than the actual width of the erect penis and has a length that is greater than (or equal to or in some cases even less than) the length of the erect penis. Of course, it is not necessary to incorporate each correlation coefficient in a size chart or in any other system or indicia or means for determining a proper condom size for a given measurement (i.e., maybe only length is measured) or measurements of an erect penis. Maybe only one correlation coefficient is utilized. Practically speaking, however, it is presently preferred that at least two correlation coefficients be used and incorporated in the size chart 81 to alter the length and width of the condom so it is greater and less than, respectively, the length and width of the erect penis that is being fitted with a condom. Incorporating at least one coefficient to calculate from measurements made on an erect penis the proper prophylactic measurements is critical in the practice of the invention and in preparing size charts 81 or other charts or conversion systems that correlate penile measurements with proper selected condom sizes.

The value of the each correlation coefficient can vary as desired. For example, the width correlation coefficient may be different for larger condoms than smaller condoms. And the particular correlation coefficient used for a particular size condom may vary depending on the properties of the latex used to make the condom. As noted, however, the width correlation coefficient will normally be less than one and the length correlation coefficient typically will be greater than one, although in some case the length correlation coefficient might equal one or be less than one and the width correlation coefficient may be equal to one.

One advantage of the invention is believed to be that it provides an inexpensive measurement kit or “fit kit”.

A second advantage of the invention is that the measurement kit can readily be obtained over the Internet. This delivery system does not require a user to interact directly with another individual, helps to maintain the privacy of the user, makes the transaction more comfortable for the user, and increases the likelihood that the user will use a condom to maximize personal safety and pleasure.

A third advantage of the invention is that it uses queries about the user's software to help confirm that dimensional parity of the measurement kit will be retained.

A fourth believed advantage of the invention is that it uses a download of software to maintain dimensional parity of the measurement kit.

A fifth advantage of the invention is that it uses queries about the user's printer to maintain dimensional parity.

A sixth important advantage of the invention is that it provides a printer adjustment chart that enables a user to adjust his printer to maintain dimensional parity.

A seventh believe advantage of the invention is that it provides a chart or other conversion means that incorporates correlation coefficient to associate penile measurements with an appropriately dimensioned condom.

An eighth advantage of the invention is that it provides a measurement that provides on a single page the necessary scales, instructions, correlation information, and order information for a user.

A ninth advantage of the invention is that it provides a measurement kit that is readily incorporated in conventional condom packets or containers.

A tenth perceived advantage of the invention is that the measurement kit provides pictorial as well as verbal instructions.

An eleventh perceived advantage of the invention is that it provides improvements in an area of the sciences—i.e. condoms—that has been relatively static and resistant to change for decades, if not centuries.

Having described my invention in such terms as to enable those of skill in the art to make and practice it, and having described the presently preferred embodiments thereof, I Claim:

1. A method for delivering by a computer network a prophylactic sizing kit to a remote site that is remote from a point of sale of prophylactics, comprising the step of
(a) providing a printable prophylactic sizing kit including
(i) at least one downloadable scale for measuring at least one of a pair comprising the length and width of an erect penis,
(ii) instructions for utilizing said scale,
(iii) a downloadable size chart correlating measurements made with said scale with indicia utilized to provide prophylactics of a selected size;
(b) providing a computer network web site to
(i) identify particular word processing software to download said prophylactic sizing kit,
(ii) identify a printer that alters the size of said sizing kit when said sizing kit is downloaded form said web site,
(iii) provide a downloadable printer adjustment chart when said printer is used to download said sizing kit from said web site,
(iv) provide downloadable instructions for utilizing said printer adjustment chart;
(c) downloading to said remote site from said web site said sizing kit, printer adjustment chart, said instructions to utilizing said scale, and said instructions for utilizing said printer adjustment chart;
(d) utilizing said instructions for utilizing said printer adjustment chart to determine if said scale in said sizing kit is properly sized; and,
(e) when said scale is not properly sized, using said printer adjustment chart to adjust said printer to print said sizing kit with a properly sized scale.

2. A method for delivering by a computer network a prophylactic sizing kit to a remote site that is remote from a point of sale of prophylactics and for obtaining a prophylactic from the point of sale, comprising the step of
(a) providing a printable prophylactic sizing kit including
(i) at least one downloadable scale for measuring at least one of a pair comprising the length and width of an erect penis,
(ii) instructions for utilizing said scale,
(iii) a downloadable size chart correlating measurements made with said scale with indicia utilized to provide prophylactics of a selected size;
(b) providing a computer network web site to
(i) identify particular word processing software to download said prophylactic sizing kit such that said software will maintain the size o the document when the document is downloaded from the web site,
(ii) identify a printer that will maintain the proper size of said document when said document is downloaded from said web site;
(c) downloading to said remote site from said web site said sizing kit, and said instructions to utilize said scale;
(d) utilizing said printer to print said sizing kit;
(e) utilizing said instructions to measure at least one of said pair at said remote site;
(f) utilizing said size chart to correlate the measurements of at least one of said pair with a prophylactic of a selected size; and,
(g) ordering said prophylactic of said selected size from said point of sale.

3. A method for delivering a prophylactic sizing kit to a remote site that is remote from a point of sale of prophylactics and for obtaining a prophylactic from the point of sale, comprising the step of
(a) providing digital data defining a printable prophylactic sizing kit document including
(i) at least one scale for measuring comprising at least one of a pair comprising the length and width of an erect penis,
(ii) instructions for utilizing said scale,
(iii) a size chart correlating measurements made with said scale with indicia utilized to provide prophylactics of a selected size;
(b) providing a computer program to download said sizing kit document to a printer such that said software will maintain the proper size of said document when the document is downloaded to said printer;
(c) providing a printer that maintains the proper size of said document when said document is printed by said printer;
(d) utilizing said computer program to download said sizing kit document to said printer such that said printer prints said prophylactic sizing kit;
(e) delivering said printed prophylactic sizing kit to said remote site;
(f) utilizing said instructions to measure at least one of said pair at said remote site;
(g) utilizing said size chart to correlate the measurements of at least one of said pair with a prophylactic of a selected size; and,
(h) ordering said prophylactic of said selected size from said point of sale.

4. Apparatus to deliver a prophylactic sizing kit to a remote site that is remote from a point of sale of prophylactics and for obtaining a prophylactic from the point of sale, comprising
(a) digital data defining a printable prophylactic sizing kit document including
(i) at least one scale for measuring at least one of a pair comprising the length and width of an erect penis,
(ii) instructions for utilizing said scale,
(iii) a size chart correlating measurements made with said scale with indicia utilized to provide prophylactics of a selected size;
(b) a computer program to download said sizing kit document to a printer such that said software will maintain the proper size of said document when the document is downloaded to said printer;
(c) a printer
(i) to print said sizing kit document, and
(ii) maintain the proper size of said document when said document is printed by said printer;
(d) a system to deliver said printed prophylactic sizing kit to said remote site;
(e) a system to order a prophylactic of a selected size from said point of sale after
(i) said size kit and said instructions are used to measure at least one of said pair at said remote site, and
(ii) said size chart is utilized to correlate the measurements of at least one of said pair with a prophylactic of a selected size.

5. Apparatus to deliver a prophylactic sizing kit and prophylactic to a site for use to obtain additional fitted prophylactics from a point of sale based upon measurements of an erect penis taken with the sizing kit, comprising
(a) a container;
(b) a prophylactic sizing kit in said container to measure the erect penis and indicate a preferred condom having a shape and dimension that permits said preferred condom to closely fit the erect penis, said kit including
(i) at least one scale for measuring at least one of a pair comprising the length and width of the erect penis,
(ii) instructions for utilizing said scale,
(iii) a size chart correlating measurements of the erect penis made with said scale with indicia utilized to identify a fitted penile condom having a selected shape and dimension, said size chart including at least one correlation coefficient to adjust at least one flat measurement of the fitted condoms to be different from the corresponding measurement of the erect penis such that said fitted penile condom more securely fits the erect penis;
(c) a system to deliver said container to said site; and,
(d) a system to order a fitted penile condom of a selected shape and dimension from said point of sale after
(i) said size kit and said instructions are used to measure at least one of said pair at said site, and
(ii) said size chart is utilized to correlate the measurements of at least one of said pair with a selected fitted prophylactic.
